(19) United States
(12) Patent Application Publication
(10) Pub. No.: US 2010/0010431 A1
(43) Pub. Date: Jan. 14, 2010

(54) Multi Lumen Suction Irrigator

(76) Inventor: Matthew John Tulley, Holmfirth (GB)

Correspondence Address: JAMES C. WRAY, 1493 CHAIN BRIDGE ROAD, SUITE 300, MCLEAN, VA, 22101, US

(21) Appl. No.: 11/922,168

(22) PCT Filed: Jun. 9, 2006
(86) PCT No.: PCT/GB2006/002106

§ 371 Date: Dec. 13, 2007

(30) Foreign Application Priority Data
Jun. 13, 2005 (GB) 0511928.4

(51) Int. Cl. A61M 1/00

(52) U.S. Cl. 604/35

(57) ABSTRACT

A multi-lumen tube (1) is used to supply the operating site during laparoscopic surgery. Flow controls in the handle allow independent operation of suction (5) and irrigation (6); none, one at a time, or both together. Having separate flow channels to the distal end of the suction irrigator allows the irrigation flow to be optimised for cleaning performance using high pressure low flow. Nozzle performance is ensured by design of flow areas and geometry, and adequate control by selection of irritation flow control areas. A bypass valve also allows high irrigation flows to be supplied via the suction lumen.

The present disclosure generally relates to multi lumen suction irrigators used in laparoscopic surgery

### BACKGROUND

Suction irrigators are an important tool used during laparoscopic (keyhole) surgery. They are used both to supply irrigation to the surgical site to wash and to suck from the site, eg to remove debris, blood, other fluids and solids. They are particularly useful towards the end of procedures to identify the site of any bleeding prior to haemostasis. They are usually used through a 5 mm diameter patient access port—the port maintains pressure inside the abdomen during surgery.

Traditional suction irrigators comprise a single lumen tube (usually 5 mm OD) and a valve that can connect the tube to either suction or irrigation. They are typically made from stainless steel, and are cleaned and sterilised between patients. The valves have to be dismantled for effective cleaning, even so they are difficult to clean because of valve and tube geometry. The valves can leak as they typically have no resilient seals (they would not withstand repeated autoclaving).

To overcome the cleaning and leakage problems, several single use suction irrigators have been developed. These generally operate in the same manner as the reusable instruments, but are clean (being single use) and do not leak (resilient seals used). They all use a single lumen tube to both supply irrigation and suction. The tube is a large diameter to fit the access port and provide adequate suction for blood clots, tissue and volume of liquid. When used for irrigation, the flow of liquid is generally low energy and flows slowly from the tube. Some companies provide additional pressure to the irrigation, either from a disposable pump or a pressurised vessel (eg Nitrogen pressurised). While this gives better washing the high flow rates consume a lot of irrigation fluid requiring frequent suction. Pressures also have to be relatively low to control the total energy in the fluid stream.

Sharing one lumen for both suction and irrigation also results in the ‘dirty’ fluid recently sucked up being returned to the patient—making detection of bleeding more difficult The proposal therefore includes two flow paths; one for suction and one for irrigation. The suction flow path has a large distal end to allow for effective suction, and the irrigation flow path ends with a nozzle to provide a jet of irrigation, similar to a shower. It thereby always washes with clean fluid, and a smaller quantity of fluid is used, but a more effective wash is provided. Higher pressures can be safely used with the smaller jet as total fluid stream energy is low with low flows.

Because solid items are sometimes removed during suction, the suction flow path should be as straight as possible to avoid blockages. Most alternative designs on the market do not have a straight or smooth flow path through the suction valve and so can block in use.

Sometimes it is necessary to deliver significant volumes of liquid into the abdomen, eg anti-adhesion fluid at the end of the procedure. The high pressure low flow irrigation jet is not suitable for this as it would take too long to deliver the fluid. A bypass valve is therefore proposed that allows the irrigation flow to pass down both the irrigation jet and also the large suction lumen into the patient.

Control of both the suction and irrigation flows is important for the surgeons, and so the valves in the handle have to be capable of varying the flow smoothly up to maximum levels. The ergonomics of holding traditional Suction Irrigators are often not comfortable; requiring high finger forces, an uncomfortable hand and arm position, and leading to fatigue and poor fluid control.

The proposed device splits controls between fingers and thumb, and enables improved ergonomics by use of a pistol grip layout.

### SUMMARY OF THE DISCLOSURE

The multi lumen suction irrigator comprises a twin lumen tube connected to a handle with controls for suction and irrigation. This allows suction and irrigation to be used independently; either none, one at a time, or both together. The handle connects to a standard operating theatre suction outlet, and to a source of high pressure irrigation fluid.

The irrigation fluid is supplied through a nozzle(s) at the distal end of the twin lumen to give a jet, which compared to a traditional suction irrigator is higher pressure and lower flow. The valve to control the irrigation nozzle has a control characteristic suitable for effective control of this high pressure low flow stream. In addition a bypass valve can be opened to provide a low pressure high flow of irrigation fluid via the suction lumen.

### BRIEF DESCRIPTION OF THE DRAWINGS

FIG. 1 shows an overview of a twin-lumen suction irrigator in accordance with the invention;

FIG. 2 shows a section of the twin-lumen tube;

FIG. 3 shows the distal end of the twin-lumen tube;

FIG. 4 shows a control mechanism for the irrigation;

FIG. 5 shows a suction valve in the closed position;

FIG. 6 shows the suction valve in the open position;

FIG. 7 shows a bypass valve in the closed position;

FIG. 8 shows the bypass valve in the open position; and

FIG. 9 shows the irrigator of FIG. 1 with an enlarged suction over tube.

### DETAILED DESCRIPTION OF THE PREFERRED EMBODIMENT

As shown in FIG. 1, the twin lumen suction irrigator comprises a twin lumen rigid tube 1 that enters the patient through an access port 2 so that the distal end 3 can be used to supply irrigation or suction at the site of the procedure. Flows are controlled with a handle 4 comprising an irrigation control 5 a suction control 6 and a bypass valve 7. The handle is connected to the operating theatre suction connection 8 and a supply of pressurised sterile irrigation fluid 9 and by two flexible tubes 10.

The overall handle layout is a pistol grip design; this is more ergonomic in use, with better surgeon hand and arm positions than with the traditional ‘in line’ valve layout. The functions of suction and irrigation are shared between the fingers and the thumb to give improved control and feel. In the preferred embodiment the thumb is used for irrigation control and the fingers for suction control.

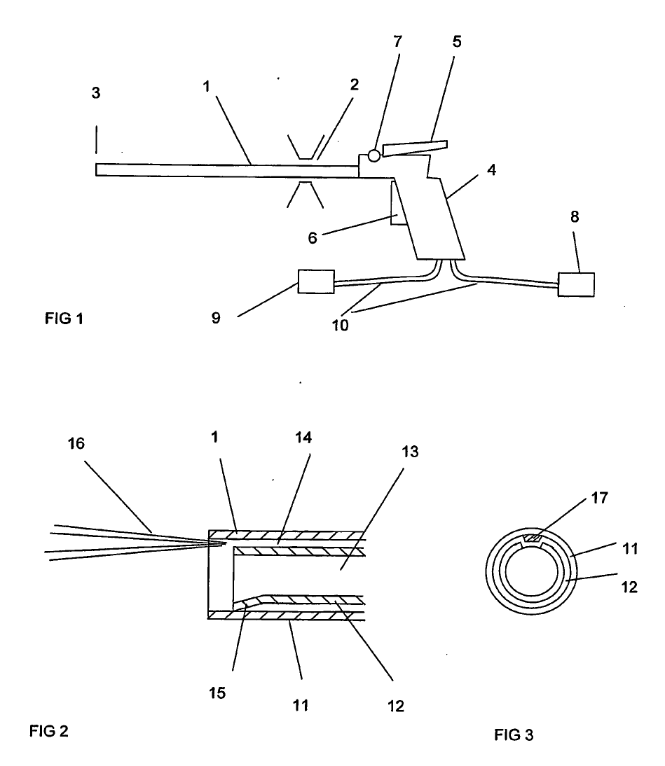

The detail of the distal end 3 of the twin lumen rigid tube 1 is shown in FIGS. 2 and 3. FIG. 2 shows a cross section of the twin lumen rigid tube comprised of an outer tube 11 and an inner tube 12. The inner tube provides a lumen for suction 13, and the space between the inner and outer tube provides a lumen for irrigation 14. The inner tube is flared at the distal end 15 to close the irrigation lumen, while a jet is formed 16 by local deformation 17 of the tube flare. This is also shown in FIG. 3, which is an end view of the tube. The outer tube 11 contains. the inner tube 12 and the nozzle formed in the inner tube flare 17 forms the jet.

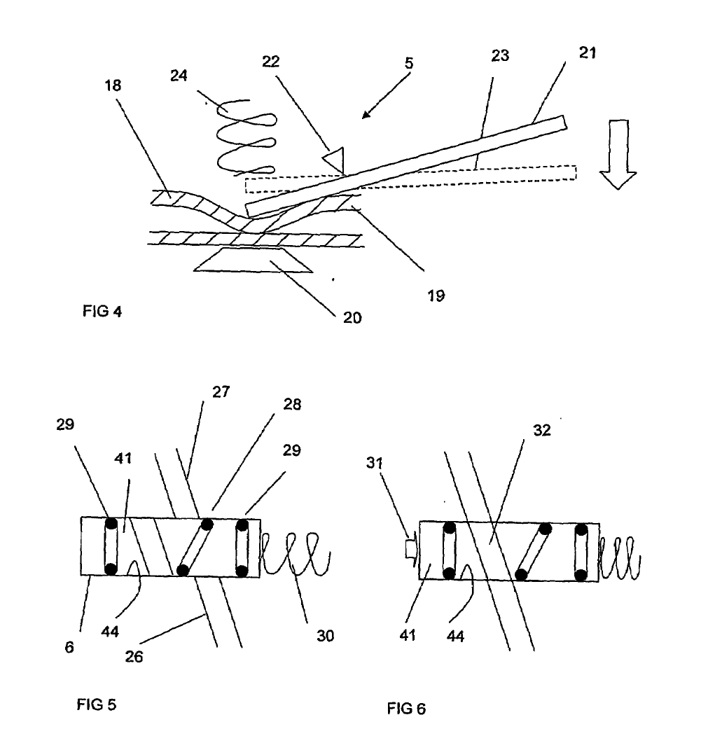

FIG. 4 shows a section through the irrigation control 5 in the handle 4. This includes a flexible flow passage tube 18 connected to the irrigation lumen 14. The flow passage 19 of the tube 18 connects to the pressurised irrigation fluid tube 9,10. The flow is controlled by squeezing the flexible flow passage between a fixed surface 20 and a lever 21 activated by, or forming, the irrigation control 5 show in FIG. 1. The lever is pivoted about fulcrum 22 to allow it to move to an open position 23 against the action of a return spring 24 which keeps the flow passage closed in normal operation. Careful selection of lever geometry and flow passage size enables effective control of irrigation flow over a wide range. Maximum flow rate is adjusted by varying the irrigation supply pressure 9.

FIGS. 5 and 6 show the suction valve 6 that comprises a circular cylindrical piston 41 slidable axially in a sleeve 44 formed in the body 4. The sleeve includes input and output for ports 26,27 of the suction lumen 13,10,8. The theatre suction 8 connects to one port 26, while the other port 27 connects to the suction lumen 13. In the closed position shown in FIG. 5 the ports are separated by an ‘O’ ring 28 around piston 41, while both of the ports are sealed from the atmosphere by ‘O’ rings 29. The valve remains closed under the action of a spring 30.

FIG. 6 shows the suction valve in the open position. The valve is moved by application of force axially 31 to a stop position where a port 32 in the piston 41 valve lines up with the other two ports 26,27. In this position the flow path is smooth and straight, thereby minimising the risk of blockage within the valve.

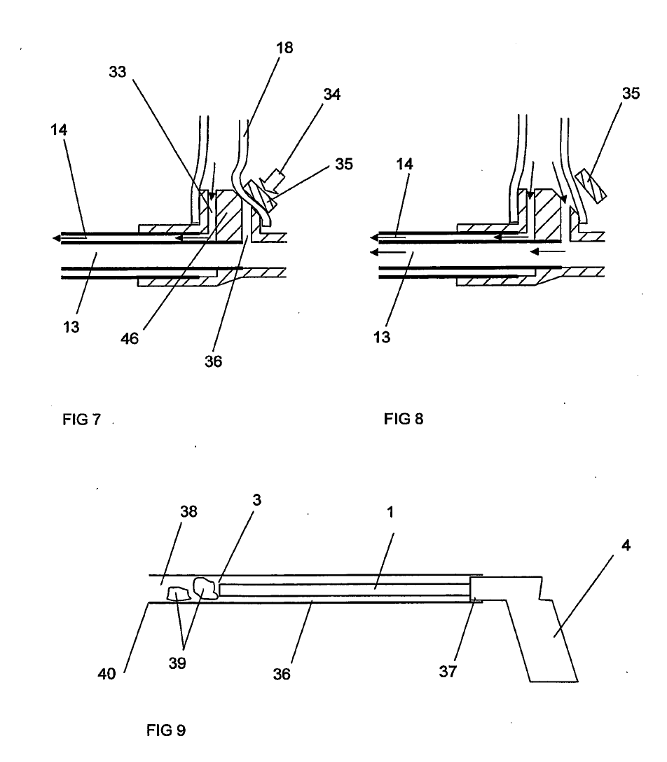

FIGS. 7 and 8 show the bypass valve 7. Irrigation supply from the tube 18 passes through a port 33 of manifold 46 into the. Irrigation lumen 14. The flow path into the suction lumen 13 is blocked by the application of a clamp force 34 onto a bypass valve 35 closing the wall of the tube over any. bypass port 36 in the manifold 46. FIG. 8 shows the bypass valve in the open position. The bypass valve 35 is moved away from the tube 18, enabling the internal pressure to open the port 36. This allows the irrigation flow 18 to access both the irrigation lumen 14 and the suction lumen 13. In this, mode, of course, the suction valve 6 is kept closed.

FIG. 9 shows an over tube 36 fitted on the outside of the rigid tube 1, being a close fit on a spigot 37 at the end of the handle 4. The tube is longer than the inner rigid tube, thereby providing a retrieval space 38 between the end 3 of the inner tube 1 and the distal end 40 of the over tube 38. This tube is designed to fit in a 10 mm diameter patient access port. In use during laparoscopic surgery, larger materials 39 can be drawn up into the retrieval space and then be withdrawn, together with the suction irrigator 1, to remove them from the patient. Outside the patient the over tube can be removed, the material discarded, and the over tube be refitted.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

1. A suction irrigator comprising:
an irrigation passage controlled by an irrigation valve and providing a relatively narrow irrigation path at an exit of the passage to form a jet of irrigation fluid when said irrigation valve is opened;
a suction passage providing a relatively wide and unconstricted path from an entrance thereof to a vacuum source;
a suction valve to close the suction passage; and
a bypass valve to connect the irrigation passage to the suction passage between the suction valve and its entrance.

2. A suction irrigator as claimed in claim 1, in which a manifold communicates with both passages and supports a flexible tube connected thereto, the tube being provided with irrigation fluid when said irrigation valve is opened, said bypass valve comprising a closure element deforming said tube to close the communication between said tube and suction passage.

3. A suction irrigator as claimed in claim 2, in which said irrigation valve is achieved by deformation of said tube.

4. A suction irrigator as claimed in claim 1, in which said suction and irrigation passages comprise a multi-lumen tube.

5. A suction irrigator as claimed in claim 1, in which said valves are disposed on a pistol-grip handle of the irrigator, and positioned so that the irrigation valve is operable by one of the finger or thumb of a user of the irrigator holding the handle, and the suction valve is operable by the other of the finger or thumb of the user.

6. A suction irrigation system as claimed in claim 1, in which irrigation control is achieved by pinching the irrigation flow path closed.

7. A suction irrigator as claimed in claim 1, in which the suction valve provides a straight line flow path when open.

8. A suction irrigator as claimed in claim 1 in which said suction valve comprises a piston slidable in a valve cylinder against the action of a return spring, the cylinder comprising inlet and outlet ports, said inlet port being connected to said passage and said outlet port being connected to said vacuum source, a valve passage through said piston connecting said ports when registered therewith.

9. A suction irrigator as claimed in claim 8, in which an O-ring around said piston seals the piston to said cylinder and isolates said ports when said valve passage is not in register with said ports.

10. A suction irrigator as claimed in claim 9, in which said ports are stepped in relation to one another and said valve passage is inclined through the piston with respect to the axis of the cylinder.

11. A suction irrigator as claimed in claim 1, further comprising a larger diameter tube disposable around said passages to provide suction over a larger section for retrieval of larger items.

12. A suction irrigator, substantially as hereinbefore described with reference to the accompanying drawings.
